(19) United States
(12) Patent Application Publication
(10) Pub. No.: US 2008/0254518 A1
(43) Pub. Date: Oct. 16, 2008

(54) Liquefaction Processes

(75) Inventor: Swapnil Bhargava, Raleigh, NC (US)

Correspondence Address: NOVOZYMES NORTH AMERICA, INC., 500 FIFTH AVENUE, SUITE 1600, NEW YORK, NY, 10110, US

(73) Assignee: Novozymes North America, Inc., Franklinton, NC (US)

(21) Appl. No.: 10/593,165

(22) PCT Filed: Mar. 18, 2005
(86) PCT No.: PCT/US05/09321

§ 371 Date: Sep. 18, 2006

Related U.S. Application Data
(60) Provisional application No. 60/554,615, filed on Mar. 19, 2004.
(60) Provisional application No. 60/575,133, filed on May 28, 2004.

(51) Int. Cl. C12P 19/14; C12P 7/06

(52) U.S. Cl. 435/99; 435/161

(57) ABSTRACT

The present invention relates to method of liquefying starch-containing material, wherein the method is carried out in stages. The invention also relates to a process of producing ethanol comprising a liquefaction step carried out according to the liquefaction method of the invention.

### CROSS-REFERENCE TO RELATED APPLICATIONS

This application claims priority from U.S. provisional application entitled “Liquefaction Processes”, filed on Mar. 10, 2005 (serial number not yet assigned), U.S. provisional application No. 60/575,133, filed on May 28, 2004, and U.S. provisional application No. 60/554,615, filed on Mar. 19, 2004, which are hereby incorporated by reference.

### FIELD OF THE INVENTION

The present invention relates to methods of liquefying starch-containing material in stages. A liquefaction method of the invention is suitable as step in processes for producing syrups and fermentation products, such as ethanol. The invention also relates to processes for producing syrups and fermentation products, such as ethanol, comprising liquefying starch-containing material in stages in accordance with a liquefaction method of the invention.

### BACKGROUND OF THE INVENTION

Liquefaction is a well known process step in the art of producing syrups and fermentation products from starch-containing materials. During liquefaction starch is converted to shorter chains and less viscous dextrins. Generally liquefaction involves gelatinization of starch simultaneously with or followed by addition of alpha-amylase.

Often liquefaction is carried out as a three-step hot slurry process. The slurry is heated to between 60-90° C. and alpha-amylase (typically around ⅓ of the total dose) is added to initiate liquefaction (thinning). The slurry is then jet-cooked at high temperature. The slurry is then cooled and more alpha-amylase (typically around ⅔ of the total dose) is added to finalize hydrolysis (secondary liquefaction).

Even though liquefaction has already been improved significantly there is still a need for improving liquefaction suitable in syrup and fermentation product production processes.

### SUMMARY OF THE INVENTION

The present invention relates to providing methods of liquefying starch-containing material that advantageously can be included in processes for producing syrups or fermentation products. The invention also provides processes of producing fermentation products and syrups including a liquefaction method of the invention carried out in stages.

According to the first aspect the invention relates to a method of liquefying starch-containing material, wherein the method comprises the step of treating starch-containing material at a temperature above the gelatinization temperature with an alpha-amylase added in two or more stages.

In a preferred embodiment the method comprises the stages of:
(a) treating starch-containing material with an alpha-amylase at a temperature above the initial gelatinization temperature for 30-180 minutes,
(b) treating the material in step (a) in a second stage with an alpha-amylase at a temperature above the initial gelatinization temperature for a period of between 30-180 minutes,
   wherein from 20-90% of the alpha-amylase added during liquefaction steps (a) and (b) is added during step (a) and 10-80% is added during step (b).

In a preferred embodiment the alpha-amylase is of bacterial, especially Bacillus origin.

The “above the initial gelatinization temperature” means above the lowest temperature at which gelatinization of the starch commences. Starch heated in water begins to gelatinize between 55° C. and 75° C.; the exact temperature of gelatinization depends on the specific starch, and can readily be determined by the skilled artisan. Thus, the initial gelatinization temperature may vary according to the plant species, to the particular variety of the plant species as well as with the growth conditions. In the context of this invention the initial gelatinization temperature of a given starch-containing material is the temperature at which birefringence is lost in 5% of the starch granules using the method described by Gorinstein. S, and Lii. C., Starch/Stärke, Vol. 44 (12) pp. 461-466 (1992).

In a second aspect the invention provides a process of producing a fermentation product from starch-containing material by fermentation, said process comprises:
(i) liquefying starch-containing material in accordance with a liquefaction method of the invention;
(ii) saccharifying the liquefied mash obtained;
(iii) fermenting the material using a fermenting organism.

In a preferred embodiment the fermentation product is ethanol. Optionally the fermentation product is recovery after fermentation. In an embodiment the saccharification and fermentation is carried out as a simultaneous saccharification and fermentation process (SSF process).

In a final aspect the invention relates to a process of producing syrup from starch-containing material comprising the steps of:
(i) liquefying starch-containing material in accordance with the liquefaction method of the invention;
(ii) saccharifying the liquefied material.

### BRIEF DESCRIPTION OF THE INVENTION

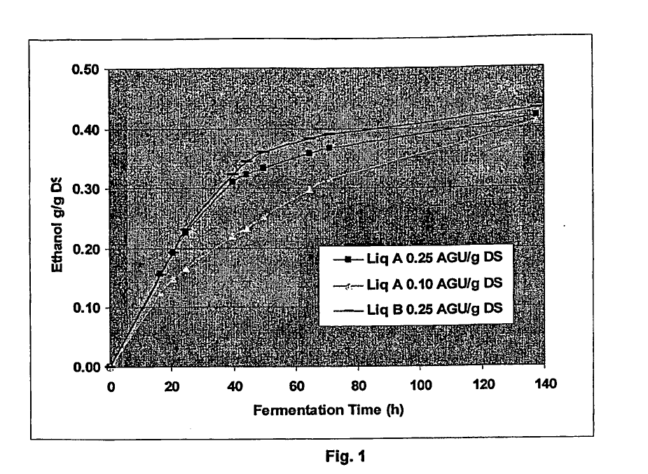

FIG. 1 shows the ethanol yields after SSF for two stage liquefaction treatments.

### DESCRIPTION OF THE INVENTION

The present invention relates to providing methods of liquefying starch-containing material. The liquefaction method of the invention may advantageously be included in processes for producing syrups or fermentation products. The invention also provides processes of producing fermentation products and syrups including a liquefaction method of the invention carried out in stages.

The present inventor found that alpha-amylase may advantageously be added at different stages during liquefaction. In Example 1 it is shown that when adding balanced amounts of alpha-amylase in two stages during liquefaction reduced amount of residual starch is observed. Staging results in better utilization of starch-containing material and improves down stream processing. Further, a higher product yield is obtained compared to single stage liquefaction where all alpha-amylase is added initially.

Without being limited to any theory it is believed that the liquefaction method of the invention is improved compared to a single stage liquefaction process because initially added alpha-amylase will gradually be inactivated or will become less active due to exposure to high temperatures for long periods of time. In other words, since the alpha-amylase in the first stage is subjected to high temperature for a long period of time, it looses its activity and is therefore not effective during the entire liquefaction. However adding additional enzyme at the second stage results in boosted activity and thereby utilization of remaining starch which is not acted upon in the first stage. Further, by adding alpha-amylase in stages there will be “fresh” enzyme present to act on the starch-containing material remaining from the previous stage. It was also found that the amount of glucoamylase needed during later saccharification or SSF could be reduced significantly. Additional released sugar consequently results in improved ethanol yield even with lower glucoamylase activity.

### Liquefaction

“Liquefaction” is a process step in which starch-containing material, such as (whole) grains, is broken down (hydrolyzed) into maltodextrins (dextrins). Before initiation of liquefaction starch-containing material is reduced in size and mixed with water to prepare an aqueous slurry. The aqueous slurry is heated to above the initial gelatinization temperature. This results in increased viscosity. In one embodiment alpha-amylase is added at this point of time to initiate thinning of the slurry. When the slurry may be jet cooked at high temperature and finally subjected to secondary liquefaction using an alpha-amylase. The invention concerns the secondary liquefaction step. However, according to the invention, no primary liquefaction, including jet-cooking, needs to take place. According to the invention liquefaction is carried out in stages where alpha-amylase is added during each stage. This makes liquefaction more efficient.

The liquefaction method of the invention may be carried out as a one, two or three-step hot slurry process where the final step is carried out in stages. In the first step the aqueous slurry may initially be heated to between 60-95° C., preferably 65-90° C. Alpha-amylase may be added to Initiate liquefaction (thinning). In the second step the slurry may be jet-cooked at a temperature between 95-140° C., preferably 105-125° C., for 1-15 minutes, preferably for 3-10 minute, especially around 5 minutes. Then the slurry is cooled and alpha-amylase is added during stages at elevated temperature during the third step. In a preferred embodiment this step is carried out in two stages.

In other words, in the first stage (referred to stage (a)) the slurry containing starch-containing material is subjected to an alpha-amylase for a suitable period of time. In the second stage (referred to as stage (b)) more alpha-amylase is added. Using the staging approach a decrease in residual starch is obtained, i.e., more starch-containing material is converted to smaller sugars. This results in higher yields of the desired final product, i.e. fermentation product or syrup.

It is to be understood that alpha-amylase may not/need not be added during the first step, i.e. prior to jet-cooking. Further, jet-cooking may not/need not be carried out. Therefore, in one embodiment no alpha-amylase is added during the first step and no jet-cooking is carried out. In other words, only the staging liquefaction method of the invention needs to be carried out according to the invention.

Consequently, in the first aspect the present invention relates to a method of liquefying starch-containing material, wherein the method comprises the step of treating starch-containing material at a temperature above the gelatinization temperature with an alpha-amylase added in two or more stages.

In a preferred embodiment the method comprises the stages of:
(a) treating starch-containing material with an alpha-amylase at a temperature above the initial gelatinization temperature for 30-180 minutes,
(b) treating the material in step (a) in a second stage with an alpha-amylase at a temperature above the initial gelatinization temperature for a period of between 30-180 minutes,
   wherein from 20-90% of the alpha-amylase added during liquefaction steps (a) and (b) is added during step (a) and 10-80% is added during step (b).

Stages (a) and (b), respectively, may be carried out in a single tank or in separate tanks. In a preferred embodiment 60-80 wt.-% of the total amount of alpha-amylase added during stages (a) and (b) is added during stage (a) and 30-50 wt.-% is added during stage (b).

In an embodiment from 0.01-0.06 wt.-% alpha-amylase per gram starch-containing material is added during stage (a) and 0.01-0.05 wt. % alpha-amylase per gram starch-containing material is added during stage (b), preferably from 0.03-0.05 wt-%, especially about 0.04 wt.-% alpha-amylase per gram starch-containing material is added during stage (a) and 0.01-0.03 wt-%, especially about 0.02 wt. % alpha-amylase per gram starch-containing material is added during stage (b).

In an embodiment from 0.01-0.1 KNU of alpha-amylase per gram dry solids (DS) is added during stage (a) and 0.01-0.06 KNU of alpha-amylase per gram DS is added during stage (b), preferably 0.03-0.06 KNU, especially about 0.05 KNU alpha-amylase per gram DS is added during stage (a) and 0.01-0.02 KNU, especially about 0.012 KNU alpha-amylase per gram DS is added during stage (b).

In a preferred embodiment stage (a) is carried out at a temperature between 60-95° C. for a period of 60-90 minutes. In a more preferred embodiment stage (a) is carried out at a temperature from 80-90° C. for a period of 60-90 minutes, especially at around 85° C. for around 75 minutes.

In a preferred embodiment stage (b) is carried out at a temperature from 60-95° C. for a period of 60-90 minutes. In a more preferred embodiment stage (b) is carried out at a temperature from 80-90° C. for a period of 60-90 minutes, preferably about 85° C. for about 75 minutes.

As mentioned above the aqueous slurry containing starch-containing material may in an embodiment be jet-cooked, preferably at a temperature between 90-120° C., more preferably around 105° C., for 1-15 minutes, even more preferably for 3-10 minute, especially around 5 minutes, before stage (a). If alpha-amylase is added prior to jet-cooking it may preferably be added in amounts between 0.01-0.03 wt-% per gram starch-containing material, preferably 0.15 wt-% alpha-amylase per gram starch-containing material. In a preferred embodiment 20-50 wt.-%, preferably 40-50 wt.-%, of the amount of alpha-amylase added in stage (a) (of the method of the invention) may preferably be added prior to jet-cooking.

It is to be understood that a method of the invention may also be carried out without a jet-cooking step and/or addition of alpha-amylase prior to stage (a) of the invention.

A liquefaction method of the invention is typically carried out at pH 4.5-6.5, in particular at a pH between 5 and 6.

The alpha-amylase may be any alpha-amylase, preferred an alpha-amylase mentioned in the section “Alpha-amylases” below.

### Starch-Containing Materials

Starch-containing material in context of the invention may be selected from the group consisting of: tubers, roots and whole grain; and any combination thereof. In an embodiment, the starch-containing material is obtained from cereals. The starch-containing material may, e.g., be selected from the groups consisting of corns, cobs, wheat, barley, cassava, sorghum, rye, milo and potatoes; or any combination thereof.

If the liquefaction method of the invention is included in a fermentation product production process of the invention, the raw starch-containing material is preferably whole grains or at least mainly whole grain. A wide variety of starch-containing whole grain crops may be used as raw material including: corn (maize), milo, potato, cassava, sorghum, wheat, and barley. Thus, in one embodiment, the starch-containing material is whole grains selected from the group consisting of corn (maize), milo, potato, cassava, sorghum, wheat, and barley; or any combinations thereof. In a preferred embodiment, the starch-containing material is whole grains selected from the group consisting of corn, wheat and barley or any combinations thereof.

The starch-containing material may also consist of or comprise a side-stream from starch processing, e.g., C₆ carbohydrate containing process streams that are not suited for production of syrups.

### Reducing the Size of Starch-Containing Material

In a preferred embodiment of the invention the size of the starch-containing material is reduced prior to liquefaction. In a preferred embodiment the material is milled. Thus, in a particular embodiment, the liquefaction method further comprises—prior to liquefaction—the step of:

i. reducing the size of starch-containing material, such as whole grains;

ii. forming a slurry comprising starch-containing material and water.

The aqueous slurry preferably contains from 10-50 wt.-%, especially 2040 wt.-% starch-containing material. The starch-containing material, such as whole grains, may in one embodiment be milled in order to open up the structure and allowing for further processing. Two processes of milling are normally used: wet and dry milling. The term “dry milling” denotes milling of the whole grains. In dry milling the whole kernel is milled and used in the remaining part of the process. Wet milling gives a good separation of germ and meal (starch granules and protein) and may typically be applied at locations where there is a parallel production of syrups. Dry milling is preferred in processes aiming at producing ethanol.

The term “grinding” is also understood as milling. In a preferred embodiment of the invention dry milling is used. Other size reducing technologies such as emulsifying technology, rotary pulsation may also be used.

### Fermentation Product Production Process

A fermentation product production process of the invention generally involves the steps of liquefaction, saccharification, fermentation and optionally recovering the fermentation product, such as ethanol, preferably by distillation.

According to this aspect, the invention relates to a process of producing a fermentation product from starch-containing material by fermentation, said process comprises:
(i) liquefying starch-containing material in accordance with the liquefaction method of the invention;
(ii) saccharifying the liquefied material;
(iii) fermenting using a fermenting organism.

The saccharification and fermentation steps may be carried out separately or simultaneously (SSF process). In a preferred embodiment of the invention the starch-containing material, such as whole grain, preferably corn, is dry milled in order to open up the structure and allow for further processing.

### Saccharification

“Saccharification” is a step in which the maltodextrin (such as material from liquefaction) is converted to low molecular sugars DP₁₋₃ (i.e., carbohydrate source) that can be metabolized by a fermenting organism, such as, yeast. Saccharification steps are well known in the art and are typically performed enzymatically using one or more carbohydrate-source generating enzymes as will be defined below. A saccharification step comprised in the process for producing, e.g., ethanol may be a well known saccharification step in the art. In one embodiment glucoamylase, alpha-glucosidase and/or acid alpha-amylase is used for treating the liquefied starch-containing material. A full saccharification step may last up to from 20 to 100 hours, preferably about 24 to about 72 hours, and is often carried out at temperatures from about 30 to 65° C., and at a pH between 4 and 6, normally around pH 4.5-5.0. However, it is often more preferred to do a pre-saccharification step, lasting for about 40 to 90 minutes, at temperature of between 30-65° C., typically about 50-60° C., followed by complete saccharification during fermentation in a simultaneous saccharification and fermentation process (SSF). SSF processes are usually carried out around the optimum temperature of the fermenting organism. In ethanol SSF processes fermentation is ongoing for 24-96 hours, such as typically 35-65 hours. In preferred embodiments, the temperature is between 26-34° C., in particular about 32° C. The pH is generally from pH 3-6, preferably around pH 4-5. The most widely used process in ethanol production is the simultaneous saccharification and fermentation process (SSF), in which there is no holding stage for the saccharification, meaning that fermenting organism, such as yeast, and enzyme(s) is(are) added together.

### Fermentation Product

The term “fermentation product” means a product produced by a process including a fermentation step using a fermenting organism. Fermentation products contemplated according to the invention include alcohols (e.g., ethanol, methanol, butanol); organic acids (e.g., citric acid, acetic acid, itaconic acid, lactic acid, gluconic acid); ketones (e.g., acetone): amino acids (e.g., glutamic acid); gases (e.g., H₂ and CO₂); antibiotics (e.g., penicillin and tetracycline); enzymes; vitamins (e.g., riboflavin, B₁₂, beta-carotene); and hormones. In a preferred embodiment the fermentation product is ethanol, e.g., fuel ethanol; drinking ethanol, i.e., potable neutral spirits; or industrial ethanol or products used in the consumable alcohol industry (e.g., beer and wine), dairy industry (e.g., fermented dairy products), leather industry and tobacco industry. Preferred beer types comprise ales, stouts, porters, lagers, bitters, malt liquors, happoushu, high-alcohol beer, low-alcohol beer, low-calorie beer or light beer. Preferred fermentation processes used include alcohol fermentation processes, as are well known in the art. Preferred fermentation processes are anaerobic fermentation processes, as are well known in the art. Where the fermentation product is ethanol it may be used as, e.g., fuel ethanol; drinking ethanol, i.e., potable neutral spirits; or industrial ethanol.

### Fermentation Organism

The term “fermenting organism” refers to any organism suitable for use in a desired fermentation process. Suitable fermenting organisms are according to the invention capable to ferment, i.e., convert sugars, such as glucose or maltose, directly or indirectly into the desired fermentation product, such as ethanol. Examples of fermenting organisms include fungal organisms, such as yeast. In an ethanol production process of the invention the fermenting organism is preferably yeast, which is applied to the saccharified material. Preferred yeast includes strains of the Saccharomyces spp., and in particular Saccharomyces cerevisiae. Commercially available yeast includes, e.g., RED STAR®/Lesaffre Ethanol Red (available from Red Star/Lesaffre, USA) FALI (available from Fleischmann's Yeast, a division of Burns Philp Food Inc., USA), SUPERSTART (available from Alltech), GERT STRAND (available from Gert Strand AB, Sweden) and FERMIOL (available from DSM Specialties).

Yeast cells are preferably applied in amounts of 10⁵ to 10¹², preferably from 10⁷ to 10¹⁰, especially 5×10⁷ viable yeast count per ml of fermentation broth. During the ethanol producing phase the yeast cell count should preferably be in the range from 10⁷ to 10¹⁰, especially around 2×10⁸. Further guidance In respect of using yeast for fermentation can be found in, e.g., “The alcohol Textbook” (Editors K. Jacques, T. P. Lyons and D. R. Kelsall, Nottingham University Press, United Kingdom 1999), which is hereby incorporated by reference.

### Recovery

According to the invention the fermentation product is optionally recovery after fermentation. Ethanol processes preferably include a step of;
(iv) distillation to obtain the ethanol;
   wherein the fermentation in step (iii) and the distillation in step (iv) is carried out simultaneously or separately/sequential; optionally followed by one or more process steps for further refinement.

### Production of Syrup

The present invention also provides a process of producing syrups from starch-containing material. Suitable starting material is exemplified in the “Starch-containing materials”-section above. The process comprises a liquefaction method of the invention followed by saccharification in order to, e.g., release sugar from the non-reducing ends of the starch or related oligo- and polysaccharide molecules in the presence of carbohydrate-source generating enzyme.

The syrup may be a syrup from the group comprising glucose (dextrose), maltose, fructose syrups, e.g., high fructose syrup (HFS), malto-oligosaccharides and isomalto-oligosaccharides or the like.

Consequently, this aspect of the invention relates to a process of producing syrup from starch-containing material, comprising
(a) liquefying starch-containing material in accordance with the liquefaction method of the invention,
(b) saccharifying the liquefied material.
   Optionally the syrup may be recovered from the saccharified material obtained in step (b).

Details on suitable liquefaction and saccharification conditions can be found above.

### Enzymes

### Alpha-Amylase

The alpha-amylase may according to the invention be of any origin. Preferred are alpha-amylases of fungal or bacterial origin.

### Bacterial Alpha-Amylases

According to the invention a bacterial alpha-amylase may preferably be derived from the genus Bacillus.

In a preferred embodiment the Bacillus alpha-amylase is derived from a strain of B. lichenifonnis, B. amyloliquefaciens, B. subtilis or B. stearothermophilus, but may also be derived from other Bacillus sp. Specific examples of contemplated alpha-amylases include the Bacillus lichenifonnis alpha-amylase (BLA) shown in SEQ ID NO: 4 in WO 99/19467, the Bacillus amyloliquefaciens alpha-amylase (BAN) shown in SEQ ID NO: 5 in WO 99/19467, and the Bacillus stearothermophilus alpha-amylase (BSG) shown in SEQ ID NO: 3 in WO 99/19467. In an embodiment of the invention the alpha-amylase is an enzyme having a degree of identity of at least 60%, preferably at least 70%, more preferred at least 80%, even more preferred at least 90%, such as at least 95%, at least 96%, at least 97%, at least 98% or at least 99% identity to any of the sequences shown as SEQ ID NOS: 1, 2, 3, 4, or 5, respectively, in WO 99/19467.

The Bacillus alpha-amylase may also be a variant and/or hybrid, especially one described in any of WO 96/23873, WO 96/23874, WO 97/41213, WO 99/19467, WO 00/60059, and WO 02/10355 (all documents hereby incorporated by reference). Specifically contemplated alpha-amylase variants are disclosed in U.S. Pat. Nos. 6,093,562, 6,297,038 or U.S. Pat. No. 6,187,576 (hereby incorporated by reference) and include Bacillus stearothermophilus alpha-amylase (BSG alpha-amylase) variants having a deletion of one or two amino acid in positions R179 to G182, preferably a double deletion disclosed in WO 1996/023873—see e.g., page 20, lines 1-10 (hereby incorporated by reference), preferably corresponding to delta(181-182) compared to the wild-type BSG alpha-amylase amino acid sequence set forth in SEQ ID NO:3 disclosed in WO 99/19467 or deletion of amino acids R179 and G180 using SEQ ID NO:3 in WO 99/19467 for numbering (which reference is hereby incorporated by reference). Even more preferred are Bacillus alpha-amylases, especially Bacillus stearothermophilus alpha-amylase, which have a double deletion corresponding to delta(181-182) and further comprise a N193F substitution (also denoted I181*+G182*+N193F) compared to the wild-type BSG alpha-amylase amino acid sequence set forth in SEQ ID NO:3 disclosed in WO 99/19467.

### Bacterial Hybrid Alpha-Amylases

A hybrid alpha-amylase specifically contemplated comprises 445 C-terminal amino acid residues of the Bacillus lichenifonnis alpha-amylase (shown as SEQ ID NO: 4 in WO 99/19467) and the 37 N-terminal amino acid residues of the alpha-amylase derived from Bacillus amyloliquefaciens (shown as SEQ ID NO: 3 in WO 99/194676), with one or more, especially all, of the following substitution:

G48A+T49I+G07A+H156Y+A181 T+N190F+I201 F+A209V+Q264S (using the Bacillus lichenifonnis numbering). Also preferred are variants having one or more of the following mutations (or corresponding mutations in other Bacillus alpha-amylase backbones): H154Y, A181T, N190F, A209V and Q264S and/or deletion of two residues between positions 176 and 179, preferably deletion of E178 and G179 (using the SEQ ID NO: 5 numbering of WO 99/19467).

### Fungal Alpha-Amylases

Fungal acid alpha-amylases include acid alpha-amylases derived from a strain of the genus Aspergillus, such as Aspergillus oryzae and Aspergillus niger alpha-amylases.

A preferred fungal alpha-amylase is a Fungamyl-like alpha-amylase which is preferably derived from a strain of Aspergillus oryzae. In the present disclosure, the term “Fungamyl-like alpha-amylase” indicates an alpha-amylase which exhibits a high identity, i.e. more than 70%, more than 75%, more than 80%, more than 85% more than 90%, more than 95%, more than 96%, more than 97%, more than 98%, more than 99% or even 100% identity to the mature part of the amino acid sequence shown in SEQ ID NO: 10 in WO 96/23874.

Another preferred acid alpha-amylase is derived from a strain Aspergillus niger. In a preferred embodiment the acid fungal alpha-amylase is the one from A. niger disclosed as “AMYA_ASPNG” in the Swiss-prot/TeEMBL database under the primary accession no. P56271 and described in more detail in WO 89/01969 (Example 3). The acid Aspergillus niger acid alpha-amylase is also shown as SEQ ID NO: 1 in WO 2004/080923 (Novozymes) which is hereby incorporated by reference. Also variants of said acid fungal amylase having at least 70% identity, such as at least 80% or even at least 90% identity, such as at least 95%, at least 96%, at least 97%, at least 98%, or at least 99% identity to SEQ ID NO: 1 in WO 2004/080923 are contemplated. A suitable commercially available acid fungal alpha-amylase derived from Aspergillus niger is SP288 (available from Novozymes A/S, Denmark).

The fungal acid alpha-amylase may also be a wild-type enzyme comprising a carbohydrate-binding module (CBM) and an alpha-amylase catalytic domain (i.e., a none-hybrid), or a variant thereof. In an embodiment the wild-type acid fungal alpha-amylase is derived from a strain of Aspergillus kawachii.

### Commercial Alpha-Amylase Products

Preferred commercial compositions comprising alpha-amylase include MYCOLASE™ (DSM, Holland), BAN™, TERMAMYL™ SC, FUNGAMYL™, LIQUOZYME™ X and SAN™ SUPER, SAN™ EXTRA L (Novozymes A/S) and CLARASE™ L-40,000, DEX-LO™, SPEZYME™ FRED, SPEZYME™ ETHYL, SPEZYME™M, and SPEZYME™ DELTA AA (Genencor Int.), and the acid fungal alpha-amylase sold under the trade name SP288 (available from Novozymes A/S, Denmark).

### Carbohydrate-Source Generating Enzyme

The term “carbohydrate-source generating enzyme” includes glucoamylase (being glucose generators), beta-amylase and maltogenic amylase (being maltose generators). A carbohydrate-source generating enzyme is capable of producing a carbohydrate that can be used as an energy-source by the fermenting organism(s) in question, for instance, when used in a process of the invention for producing a fermentation product, such as ethanol. The generated carbohydrate may be converted directly or indirectly to the desired fermentation product, preferably ethanol. According to the invention a mixture of carbohydrate-source generating enzymes may be used. Especially contemplated mixtures are mixtures of at least a glucoamylase and an alpha-amylase, especially an acid amylase, even more preferred an acid fungal alpha-amylase. The ratio between acidic fungal alpha-amylase activity (AFAU) per glucoamylase activity (AGU) (AFAU per AGU) may in an embodiment of the invention be at least 0.1, in particular at least 0.16, such as in the range from 0.12 to 0.50 or more.

### Glucoamylase

A glucoamylase used according to the invention may be derived from any suitable source, e.g., derived from a micro-organism or a plant. Preferred glucoamylases are of fungal or bacterial origin, e.g., selected from the group consisting of Aspergillus glucoamylases, in particular A. niger G1 or G2 glucoamylase (Boel et al. (1984), EMBO J. 3 (5), p. 1097-1102), or variants thereof, such as one disclosed in WO 92/00381, WO 00/04136, WO 01/04273 and WO 03/029449 (from Novozymes, Denmark, hereby incorporated by reference); the A. awamori glucoamylase (WO 84/02921), A. oryzae (Agric. Biol. Chem. (1991), 55 (4), p. 941-949), or variants or fragments thereof.

Other Aspergillus glucoamylase variants include variants to enhance the thermal stability: G137A and G139A (Chen et al. (1996), Prot. Eng. 9, 499-505); D257E and D293E/Q (Chen et al. (1995), Prot. Engng. 8, 575-582); N182 (Chen et al. (1994), Biochem. J. 301, 275-281); disulphide bonds, A246C (Fierobe et al. (1996), Biochemistry, 35, 8698-8704; and introduction of Pro residues in position A435 and S436 (Li et al. (1997), Protein Engng. 10, 1199-1204. Other glucoamylases include Athelia rolfsii (previously denoted Corticium rolfsii) glucoamylase (see U.S. Pat. No. 4,727,026 and (Nagasaka, Y. et al. (1998) Purification and properties of the raw-starch-degrading glucoamylases from Corticium rolfsii, Appl Microbiol Biotechnol 50:323-330), Talaromyces glucoamylases, in particular, derived from Talaromyces emersonii (WO 99/28448), Talaromyces leycettanus (U.S. Pat. No. Re. 32,153), Talaromyces duponti, Talaromyces thermophilus (U.S. Pat. No. 4,587,215). Bacterial glucoamylases contemplated include glucoamylases from the genus Clostridium, in particular C. thermoamylolyticum (EP 135,138), and C. thermohydrosulfuricum (WO 86/01831).

Commercially available compositions comprising glucoamylase include AMG 200 L; AMG 300 L; SAN™ SUPER, SAN™ EXTRA L, SPIRIZYME™ PLUS, SPIRIZYME™ FUEL, SPIRIZYME™ B4U and AMG™ E (from Novozymes A/S); OPTIDEX™ 300 (from Genencor Int.); AMIGASE™ and AMIGASE™ PLUS (from DSM); G-ZYME™ G900, G-ZYME™ and G990 ZR (from Genencor Int.).

Glucoamylase may in an embodiment be added in an amount of 0.005-5 AGU/g DS, more preferably between 0.01-1 AGU/g DS, such as especially around 0.1-0.5 AGU/g DS.

### Beta-Amylase

At least according to the invention the a beta-amylase (E.C 3.2.1.2) is the name traditionally given to exo-acting maltogenic amylases, which catalyze the hydrolysis of 1,4-alpha-glucosidic linkages in amylose, amylopectin and related glucose polymers. Maltose units are successively removed from the non-reducing chain ends in a step-wise manner until the molecule is degraded or, in the case of amylopectin, until a branch point is reached. The maltose released has the beta anomeric configuration, hence the name beta-amylase.

Beta-amylases have been isolated from various plants and microorganisms (W. M. Fogarty and C. T. Kelly, Progress in Industrial Microbiology, vol. 15, pp. 112-115, 1979). These beta-amylases are characterized by having optimum temperatures in the range from 40° C. to 65° C. and optimum pH in the range from 4.5 to 7. A commercially available beta-amylase from barley is NOVOZYM™ WBA from Novozymes A/S, Denmark and SPEZYME™ BBA 1500 from Genencor Int., USA.

### Maltogenic Amylase

The amylase may also be a maltogenic alpha-amylase. A “maltogenic alpha-amylase” (glucan 1,4-alpha-maltohydrolase, E.C. 3.2.1.133) is able to hydrolyze amylose and amylopectin to maltose in the alpha-configuration. A maltogenic amylase from Bacillus stearothermophilus strain NCIB 11837 is commercially available from Novozymes A/S. Maltogenic alpha-amylases are described in U.S. Pat. Nos. 4,598,048, 4,604,355 and 6,162,628, which are hereby incorporated by reference.

The maltogenic amylase may in a preferred embodiment be added in an amount of 0.05-5 mg total protein/gram DS or 0.05-5 MANU/g DS.

### Production of Enzymes

The enzymes referenced herein may be derived or obtained from any suitable origin, including, bacterial, fungal, yeast or mammalian origin. The term “derived” or means in this context that the enzyme may have been isolated from an organism where it is present natively, i.e., the identity of the amino acid sequence of the enzyme are identical to a native enzyme. The term “derived” also means that the enzymes may have been produced recombinantly in a host organism, the recombinant produced enzyme having either an identity identical to a native enzyme or having a modified amino acid sequence, e.g., having one or more amino acids which are deleted, inserted and/or substituted, i.e., a recombinantly produced enzyme which is a mutant and/or a fragment of a native amino acid sequence or an enzyme produced by nucleic acid shuffling processes known in the art. Within the meaning of a native enzyme are included natural variants. Furthermore, the term “derived” includes enzymes produced synthetically by, e.g., peptide synthesis. The term “derived” also encompasses enzymes which have been modified e.g., by glycosylation, phosphorylation, or by other chemical modification, whether in vivo or in vitro. The term “obtained” in this context means that the enzyme has an amino acid sequence identical to a native enzyme. The term encompasses an enzyme that has been isolated from an organism where it is present natively, or one in which it has been expressed recombinantly in the same type of organism or another, or enzymes produced synthetically by, e.g., peptide synthesis. With respect to recombinantly produced enzymes the terms “obtained” and “derived” refers to the identity of the enzyme and not the identity of the host organism in which it is produced recombinantly.

The enzymes may also be purified. The term “purified” as used herein covers enzymes free from other components from the organism from which it is derived. The term “purified” also covers enzymes free from components from the native organism from which it is obtained. The enzymes may be purified, with only minor amounts of other proteins being present. The expression “other proteins” relate in particular to other enzymes. The term “purified” as used herein also refers to removal of other components, particularly other proteins and most particularly other enzymes present in the cell of origin of the enzyme of the invention. The enzyme may be “substantially pure,” that is, free from other components from the organism in which it is produced, that is, for example, a host organism for recombinantly produced enzymes. In preferred embodiment, the enzymes are at least 75% (w/w) pure, more preferably at least 80%, at least 85%, at least 90%, at least 95%, at least 96%, at least 97%, at least 98%, or at least 99% pure. In another preferred embodiment, the enzyme is 100% pure.

The enzymes used according to the present invention may be in any form suitable for use in the processes described herein, such as, e.g., in the form of a dry powder or granulate, a non-dusting granulate, a liquid, a stabilized liquid, or a protected enzyme. Granulates may be produced, e.g., as disclosed in U.S. Pat. No. 4,106,991 and U.S. Pat. No. 4,661,452, and may optionally be coated by process known in the art. Liquid enzyme preparations may, for instance, be stabilized by adding stabilizers such as a sugar, a sugar alcohol or another polyol, lactic acid or another organic acid according to established process. Protected enzymes may be prepared according to the process disclosed in EP 238,216.

Even if not specifically mentioned in context of a method or process of the invention, it is to be understood that the enzyme(s) or agent(s) is(are) used in an “effective amount”.

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and de-scribed herein will become apparent to those skilled in the art from the foregoing description. Such modifications are also intended to fall within the scope of the appended claims. In the case of conflict, the present disclosure including definitions will control.

Various references are cited herein, the disclosures of which are incorporated by reference in their entireties. The present invention is further described by the following examples which should not be construed as limiting the scope of the invention.

### Materials and Methods

### Enzymes:

Bacterial Alpha-amylase A; Bacillus stearothermophilus alpha-amylase variant with the mutations: I181*+G182*+N193F disclosed in U.S. Pat. No. 6,187,576 and available on request from Novozymes A/S, Denmark.
Glucoamylase T: Glucoamylase derived from Talaromyces emersonii and disclosed as SEQ ID NO: 7 in WO 99/28448.

### Yeast:

RED STAR available from Red Star/Lesaffre, USA
Stock solution for iodine method:

### 0.1N I₂

dissolve 1.3 g I₂ and 2.0 g Kl into 100 mL Dl water

### Methods:

### Alpha-Amylase Activity (KNU)

The amylolytic activity may be determined using potato starch as substrate. This method is based on the break-down of modified potato starch by the enzyme, and the reaction is followed by mixing samples of the starch/enzyme solution with an iodine solution. Initially, a blackish-blue color is formed, but during the break-down of the starch the blue color gets weaker and gradually turns into a reddish-brown, which is compared to a colored glass standard.

One Kilo Novo alpha amylase Unit (KNU) is defined as the amount of enzyme which, under standard conditions (i.e., at 37° C.+/−0.05; 0.0003 M Ca²⁺; and pH 5.6) dextrinizes 5260 mg starch dry substance Merck Amylum solubile.

A folder EB-SM-0009.02/01 describing this analytical method in more detail is available upon request to Novozymes A/S, Denmark, which folder is hereby included by reference.

Determination of FAU activity

One Fungal Alpha-Amylase Unit (FAU) is defined as the amount of enzyme, which breaks down 5.26 g starch (Merck Amylum solubile Erg. B.6, Batch 9947275) per hour based upon the following standard conditions:

| | |
|---|---|
| Substrate | Soluble starch |
| Temperature | 37° C. |
| pH | 4.7 |
| Reaction time | 7-20 minutes |

### Determination of Acid Alpha-Amylase Activity (AFAU)

Acid alpha-amylase activity is measured in AFAU (Acid Fungal Alpha-amylase Units), which are determined relative to an enzyme standard.

The standard used is AMG 300 L (from Novozymes A/S, Denmark, glucoamylase wild-type Aspergillus niger G1, also disclosed in Boel et al. (1984), EMBO J. 3 (5), p. 1097-1102) and WO 92/00381). The neutral alpha-amylase in this AMG falls after storage at room temperature for 3 weeks from approx. 1 FAU/mL to below 0.05 FAU/mL.

The acid alpha-amylase activity in this AMG standard is determined in accordance with the following description. In this method, 1 AFAU is defined as the amount of enzyme, which degrades 5.260 mg starch dry matter per hour under standard conditions.

Iodine forms a blue complex with starch but not with its degradation products. The intensity of color is therefore directly proportional to the concentration of starch. Amylase activity is determined using reverse colorimetry as a reduction in the concentration of starch under specified analytic conditions.

$$Starch + Iodine {{\to }^{Alpha \text{-} amylase}}_{40 ° C . , pH 2.5} Dextrins + Oligosaccharides$$

$$Blue / violet t = 23 sec . Decoloration$$

Standard conditions/reaction conditions: (per minute)

| | |
|---|---|
| Substrate: | Starch, approx. 0.17 g/L |
| Buffer: | Citate, approx. 0.03 M |
| Iodine (I₂): | 0.03 g/L |
| CaCl₂: | 1.85 mM |
| pH: | 2.50 ± 0.05 |
| Incubation temperature: | 40° C. |
| Reaction time: | 23 seconds |
| Wavelength: | lambda = 590 nm |
| Enzyme concentration: | 0.025 AFAU/mL |
| Enzyme working range: | 0.01-0.04 AFAU/mL |

If further details are preferred these can be found in EB-SM-0259.02/01 available on request from Novozymes A/S, Denmark, and incorporated by reference.

### Acid Alpha-Amylase Units (AAU)

The acid alpha-amylase activity can be measured in AAU (Acid Alpha-amylase Units), which is an absolute method. One Acid Amylase Unit (AAU) is the quantity of enzyme converting 1 g of starch (100% of dry matter) per hour under standardized conditions into a product having a transmission at 620 nm after reaction with an iodine solution of known strength equal to the one of a color reference.

Standard conditions/reaction conditions:

| | |
|---|---|
| Substrate: | Soluble starch. Concentration approx. 20 g |
| | DS/L. |
| Buffer: | Citrate, approx. 0.13 M, pH = 4.2 |
| Iodine solution: | 40.176 g potassium iodide + 0.088 g iodine/L |
| City water | 15°-20°dH (German degree hardness) |
| pH: | 4.2 |
| Incubation temperature: | 30° C. |
| Reaction time: | 11 minutes |
| Wavelength: | 620 nm |
| Enzyme concentration: | 0.13-0.19 AAU/mL |
| Enzyme working range: | 0.13-0.19 AAU/mL |

The starch should be Lintner starch, which is a thin-boiling starch used in the laboratory as colorimetric indicator. Lintner starch is obtained by dilute hydrochloric acid treatment of native starch so that it retains the ability to color blue with iodine. Further details can be found in EP0140410B2, which disclosure is hereby included by reference.

### Glucoamylase Activity (AGI)

Glucoamylase (equivalent to amyloglucosidase) converts starch into glucose. The amount of glucose is determined here by the glucose oxidase method for the activity determination. The method described in the section 76-11 Starch—Glucoamylase Method with Subsequent Measurement of Glucose with Glucose Oxidase in “Approved methods of the American Association of Cereal Chemists”. Vol. 1-2 AACC, from American Association of Cereal Chemists, (2000); ISBN: 1-891127-12-8.

One glucoamylase unit (AGI) is the quantity of enzyme which will form 1 micromol of glucose per minute under the standard conditions of the method.

Standard conditions/reaction conditions:

| | |
|---|---|
| Substrate: | Soluble starch. |
| | Concentration approx. 16 g dry matter/L. |
| Buffer: | Acetate, approx. 0.04 M, pH = 4.3 |
| pH: | 4.3 |
| Incubation temperature: | 60° C. |
| Reaction time: | 15 minutes |
| Termination of the reaction: | NaOH to a concentration of approximately |
| | 0.2 g/L (pH~9) |
| Enzyme concentration: | 0.15-0.55 AAU/mL |

The starch should be Lintner starch, which is a thin-boiling starch used in the laboratory as calorimetric indicator. Lintner starch is obtained by dilute hydrochloric acid treatment of native starch so that it retains the ability to color blue with iodine.

### Glucoamylase Activity (AGU)

The Novo Glucoamylase Unit (AGU) is defined as the amount of enzyme, which hydrolyzes 1 micromole maltose per minute under the standard conditions 37° C., pH 4.3, substrate: maltose 23.2 mM, buffer: acetate 0.1 M, reaction time 5 minutes.

An autoanalyzer system may be used. Mutarotase is added to the glucose dehydrogenase reagent so that any alpha-D-glucose present is turned into beta-D-glucose. Glucose dehydrogenase reacts specifically with beta-D-glucose in the reaction mentioned above, forming NADH which is determined using a photometer at 340 nm as a measure of the original glucose concentration.

| | |
|---|---|
| AMG incubation: | |
| Substrate: | maltose 23.2 mM |
| Buffer: | acetate 0.1 M |
| pH: | 4.30 ± 0.05 |
| Incubation temperature: | 37° C. ± 1 |
| Reaction time: | 5 minutes |
| Enzyme working range: | 0.5-4.0 AGU/mL |
| Color reaction: | |
| GlucDH: | 430 U/L |
| Mutarotase: | 9 U/L |
| NAD: | 0.21 mM |
| Buffer: | phosphate 0.12 M; 0.15 M NaCl |
| pH: | 7.60 ± 0.05 |
| Incubation temperature: | 37° C. ± 1 |
| Reaction time: | 5 minutes |
| Wavelength: | 340 nm |

A folder (EB-SM-0131.02/01) describing this analytical method in more detail is available on request from Novozymes A/S, Denmark, which folder is hereby included by reference.

### Determination of Maltogenic Amylase Activity (MANU)

One MANU (Maltogenic Amylase Novo Unit) may be defined as the amount of enzyme required to release one micro mole of maltose per minute at a concentration of 10 mg of maltotriose (Sigma M 8378) substrate per ml of 0.1 M citrate buffer, pH 5.0 at 37° C. for 30 minutes.

### Standard Iodine Method

boil small aliquot (10-20 mLs) of liquefied material in a test tube for several minutes

cool in ice bath

add 10-12 drops of the iodine solution

mix and let sample sit in ice water for about 10 minutes

### Determination of DE (Dextrose Equivalent)

The DE value is measured using Fehlings liquid by forming a copper complex with the starch using pure glucose as a reference, which subsequently is quantified through iodometric titration. DE (dextrose equivalent) is defined as the amount of reducing carbohydrate (measured as dextrose-equivalents) in a sample expressed as w/w % of the total amount of dissolved dry matter. It may also be measured by the neocuproine assay (Dygert, Li Floridana (1965) Anal. Biochem. No 368). The principle of the neocuproine assay is that CuSO₄ is added to the sample, Cu²⁺ is reduced by the reducing sugar and the formed neocuproine complex is measured at 450 nm.

### EXAMPLES

### Example 1

### Ethanol Yields from Two Stage Liquefaction

To investigate the effect of staging enzyme addition during liquefaction processes, liquefactions were carried out under the following conditions:

Liq A: 0.04 wt.-% BAAA of corn, 85° C., 90 Minutes,
Liq B: 0.04 wt.-% BAAA of corn, 85° C., 90 Minutes, +0.02 wt.-% BAAA, 85° C., 90 Minutes

Initially ground corn was used to make a 30 wt.-% slurry with tap water. The pH in all liquefactions was adjusted to 5.8 using diluted H₂SO₄/NaOH. Once the pH was adjusted, constant dose (0.04% wt.-% of corn or approximately 50 NU per gram DS) of Bacterial Al-pha-Amylase A (BAAA) was added to the corn mash. Slurry was then subjected to temperature condition indicated above. In case of Liq B, additional amount of enzyme was added after the initial 90 minutes.

Once the liquefaction was complete, the liquefied material was subjected to simultaneous saccharification and fermentation (SSF) after cooling down to room temperature. The effect of liquefaction treatment on SSF was evaluated via mini-scale fermentations where SSF was carried out in 16 mL polystyrene tubes. Tubes were dosed with purified Glucoamylase T for saccharification with 3 different dosages, i.e., 0.25 and 0.1 AGU/g DS for Liq A as controls and single dose (0.25 AGU/g DS) for Liq B.

After dosing the tubes with enzyme, they were inoculated with 0.04 mLg mash of yeast (RED STAR™) propagate that had been grown for 21 hours on corn mash. Vials were capped with a screw on lid which had been punctured with a very small needle to allow gas release and vortexed briefly before weighing and incubation at 32° C. Fermentation progress was followed by weighing the tubes over time. Tubes were vortexed briefly before each weighing. Weight loss values were converted to ethanol yield (g ethanol/g DS) by the following formula:

$$g ethanol / g DS = \frac{\begin{matrix}g {CO}_{2} weight loss \times \frac{1 mol {CO}_{2}}{44.0098 g {CO}_{2}} \times \\ \frac{1 mol ethanol}{1 mol {CO}_{2}} \times \frac{46.094 g ethanol}{1 mol ethanol}\end{matrix}}{g corn in tube \times \% DS of corn}$$

Liquefaction samples from both treatments were analyzed for sugar profiles using HPLC. The profiles in Table I show that adding additional alpha-amylase in Liq B resulted in more sugar release compared to the Liq A. Approximately 6% increase in overall sugar was observed in Liq B. In addition, a higher DE value was obtained in Liq B.

TABLE 1
Sugar profiles for Liquefaction treatments using HPLC

| | Dextrin | Maltotriose | Maltose | Glucose | |
|---|---|---|---|---|---|
| Liquefaction | (DP4) g/L | (DP3) g/L | (DP2) g/L | g/L | DE Value |
| Liq A | 215.022 | 31.112 | 24.516 | 12.006 | 14.94 |
| Liq B | 227.468 | 34.895 | 28.254 | 15.478 | 19.59 |

Ethanol yields from fermentation for the staging study were analyzed by weight loss due to CO₂ release (FIG. 1). The results show that Liq B produced ethanol at a faster rate than the 85° C. Liq B in the initial fermentation phase. Also weight loss data shows that Liq B treatment results in relatively higher ethanol yields compared to all the dosages for Liq A. This shows that addition of alpha-amylase in liquefaction results in not only improved ethanol yields but also in reduced need for glucoamylase dosages in SSF.

Similar observations were seen when final SSF samples (approx. 70 hours) were subjected to HPLC (Table 2). The data shows that adding extra Bacterial Alpha-Amylase A (BAAA) resulted in significantly higher ethanol yields.

TABLE 2

| | DP4+ | DP3 | DP2 | Glucose | Glycerol | Ethanol |
|---|---|---|---|---|---|---|
| SSF treatment | (g/L) | (g/L) | (g/L) | (g/L) | (g/L) | (g/L) |
| Liq A 0.25 | 8.728 | 1.628 | 1.574 | 1.268 | 14.183 | 104.853 |
| AGU/g DS | | | | | | |
| Liq A 0.1 | 25.106 | 6.083 | 2.401 | 1.982 | 12.475 | 94.867 |
| AGU/g DS | | | | | | |
| Liq B 0.25 | 7.703 | 1.777 | 2.046 | 1.689 | 14.258 | 111.942 |
| AGU/g DS | | | | | | |

1. A method of liquefying starch-containing material, wherein the method comprises the step of treating starch-containing material at a temperature above the gelatinization temperature with an alpha-amylase added in two or more stages

2. A method of claim 1, wherein the method comprises the stages of:
(a) treating starch-containing material with an alpha-amylase at a temperature above the initial gelatinization temperature for 30-180 minutes,
(b) treating the material in step (a) in a second stage with an alpha-amylase at a temperature above the initial gelatinization temperature for a period of between 30-180 minutes,
wherein from 20-90% of the total alpha-amylase added during liquefaction stages (a) and (b) is added during stage (a) and 10-80% is added during stage (b).

3. The method of claim 2, wherein 60-80% of the alpha-amylase added during liquefaction is added during stage (a) and 30-50% is added during stage (b).

4. The method of claim 2, wherein 0.01-0.06 wt.-% alpha-amylase per gram starch-containing material is added during stage (a) and 0.01-0.05 wt. % alpha-amylase per gram starch-containing material is added during stage (b).

5. The method of claim 2, wherein the temperature during stage (a) is between 60-95° C. for between 60-90 minutes.

6. (canceled)

7. The method of claim 2, wherein stage (b) is carried out at a temperature between 60-95° C. for 60-90 minutes.

8. (canceled)

9. The method of claim 2, wherein the starch-containing material is jet-cooking at 90-120° C., for 1-15 minutes before stage (a).

10. The method of claim 9, wherein 0.01-0.03 wt-% alpha-amylase per grams starch-containing material is added during jet-cooking.

11. The method of claim 9, wherein 20-50 wt-% of the amount of alpha-amylase added in stage (a) is added during jet-cooking.

12. (canceled)

13. The method of claim 1, wherein the starch-containing material is selected from the group consisting of corn, cob, wheat, barley, rye, milo and potatoes; or any combination of these.

14-16. (canceled)

17. The method of claim 2, further comprising prior to stage (a) and/or jet cooking the steps of;
i) reducing the size of starch-containing material;
ii) forming a slurry comprising the starch-containing material and water.

18. The method of claim 17, wherein the milling step is a dry milling step.

19. (canceled)

20. The method claim 1, wherein the alpha-amylase is of bacterial or fungal origin.

21. The method of claim 20, wherein the bacterial alpha-amylase is a Bacillus alpha-amylase.

22. The method of claim 1, wherein the pH during liquefaction is between about 4.5 and 7.

23. The method of claim 2, wherein 0.01-0.1 KNU of alpha-amylase per gram DS is added during stage (a) and 0.01-0.06 KNU alpha-amylase per gram DS is added during stage (b).

24. A process of producing a fermentation product from starch-containing material by fermentation, said process comprises:
(i) liquefying starch-containing material as defined in claim 1;
(ii) saccharifying the liquefied material;
(iii) fermenting the material using a fermenting organism.

25. The process of claim 24, wherein the fermentation product is ethanol.

26. (canceled)

27. The process of claim 24, wherein the saccharification and fermentation is carried out separately or simultaneously (SSF process).

28. The process of claim 27, wherein SSF is carried out for between 20 and 100 hours, preferably about 24 to 72 hours.

29. (canceled)

30. The process of claim 24, wherein the fermenting organism is Saccharomyces cerevisiae.

31. The process of claim 24, wherein the saccharification and/or fermentation is carried out in the presence of a carbohydrate-source generating enzyme.

32. The process of claim 31, wherein the carbohydrate-source generating enzyme is a glucoamylase derived from a strain of Aspergillus or a strain of Talaromyces or a strain of Athelia.

33. The process of claim 31, wherein the carbohydrate-source generating enzyme is a glucoamylase, maltogenic amylase, beta-amylase, or a combination thereof.

34. The process of claim 33, wherein the carbohydrate-source generating enzyme is in a concentration of 0.005-5 AGU/g DS.

35. A process of producing a syrup from starch-containing material comprising the steps of:
(i) liquefying starch-containing material as defined in claim 1;
(ii) saccharifying the liquefied material.

36-37. (canceled)

38. The method of claim 4, wherein 0.03-0.05 wt-% alpha-amylase per gram starch-containing material is added during stage (a) and 0.01-0.03 wt-% alpha-amylase per gram starch-containing material is added during stage (b)

39. The method of claim 21, wherein the alpha-amylase is derived from Bacillus stearothermophilus alpha-amylase or a variant thereof.

40. The method of claim 39, wherein the alpha-amylase derived from Bacillus stearothermophilus has the following mutations: R179*+G180* or I181*+G182*.

41. The method of claim 40, wherein the alpha-amylase derived from Bacillus stearothermophilus has the following mutation I181*+G182*+N193F.

42. The method of claim 41, wherein the glucoamylase is derived from a strain of Aspergillus niger, Talaromyces emersonii or Athelia rolfsii.
